(19) United States
(12) Patent Application Publication
(10) Pub. No.: US 2019/0120049 A1
(43) Pub. Date: Apr. 25, 2019

(54) Universal Downhole Fluid Analyzer With Generic Inputs

(71) Applicant: HALLIBURTON ENERGY SERVICES, INC., Houston, TX (US)

(72) Inventors: Dingding Chen, Tomball, TX (US); Bin Dai, Spring, TX (US); Christopher M. Jones, Houston, TX (US); Darren Gascooke, Houston, TX (US)

(21) Appl. No.: 15/559,800

(22) PCT Filed: Nov. 4, 2016
(86) PCT No.: PCT/US2016/060586

§ 371 Date: Sep. 19, 2017

(51) Int. Cl. E21B 49/08; G01N 33/28

(52) U.S. Cl. CPC E21B 49/08; E21B 49/00; E21B 2049/085; G01N 33/2823

(57) ABSTRACT

System and methods for downhole fluid analysis are provided. Measurements are obtained from one or more downhole sensors along a current section of wellbore within a subsurface formation. The measurements obtained from the one or more downhole sensors are transformed into principal spectroscopy component (PSC) data. At least one fluid composition or property is estimated for the current section of the wellbore, based on the PSC data and a fluid analysis model. The fluid analysis model is refined for one or more subsequent sections of the wellbore within the subsurface formation, based at least partly on the fluid composition or property estimated for the current section of the wellbore.

### FIELD OF THE DISCLOSURE

The present disclosure relates generally to fluid analysis in hydrocarbon recovery operations, and particularly, to fluid analysis based on measurements collected using downhole sensors during hydrocarbon recovery operations.

### BACKGROUND

A variety of downhole tools may be used within a wellbore in connection with analyzing downhole fluids during hydrocarbon recovery operations conducted at the wellsite. Optical sensor devices coupled to a downhole tool disposed within the wellbore can be utilized to collect measurements of multiple fluid samples taken from inside the wellbore and surrounding formation throughout the hydrocarbon recovery process. Such measurements may be used to characterize the compositions and properties of different types of downhole fluids. For example, such measurements may be applied to a fluid characterization model to evaluate different properties of interest. The model is typically calibrated on selected fluid samples from a standard fluid library under stabilized conditions using a number of predetermined parameters as model inputs that may have been derived from, or simulated with, particular detector outputs of an optical sensor. Data prediction using standard calibration inputs is usually accurate on training samples utilized for model development. However, problems in predicting formation fluid composition can arise due to field data being out of calibration data range, optical signal intensity variation with severe environment and tool conditioning, and one or more optical elements that fail to operate properly.

### BRIEF DESCRIPTION OF THE DRAWINGS

FIG. 1A is a diagram of an illustrative well system for downhole fluid analysis based on principal spectroscopy component (PSC) data obtained through optical sensor data transformation during wellsite operations.

FIG. 1B is a diagram of an illustrative wireline system for downhole fluid analysis based on PSC data.

FIGS. 2A, 2B, and 2C are plot graphs of illustrative data relationships between normalized PSC signal intensity and fluid composition or property.

FIG. 3 is a diagram of an illustrative multi-input and single-output (MISO) neural network structure for estimating a particular fluid composition or property (FCP).

FIG. 4 is a diagram of another illustrative MISO neural network structure for estimating/simulating an optical sensor response to fluids that may be measured downhole.

FIG. 5A is a plot graph showing an example of PSC data for a cluster of fluids from which a fluid sample under multiple measurement conditions may be selected as a reference fluid.

FIG. 5B is a plot graph showing measured and simulated optical responses for the fluids of FIG. 5A within a sensor parameter space.

FIGS. 6A and 6B are plot graphs showing a comparison between calibration data of original reference fluids and additional or extended fluids.

FIG. 7 is a diagram of an illustrative multi-input and multi-output (MIMO) neural network structure for converting optical sensor data into PSC data.

FIGS. 8A and 8B are cross-plots showing calibration results of reverse transformation on two PSC outputs with four sensors by using reference fluids alone.

FIGS. 9A and 9B are cross-plots of illustrative calibration results of the reverse transformation model for the same outputs of the reverse transformation of FIGS. 8A and 8B, except with a single sensor and extended data inputs.

FIG. 10A is a plot graph showing examples of different classifications for grouping fluid PSC data into a fluid type cluster.

FIG. 10B is a plot graph of PSC data relative to a cluster kernel distance associated with different classifications applied to the fluids of FIG. 10A.

FIGS. 11A, 11B, 11C and 11D are plot graphs of illustrative member network predictions on the chemical concentration of the fluids in FIGS. 10A and 10B.

FIG. 12A is a plot graph of neural network ensemble (NNE) predictions on saturates, aromatics, resins and asphaltanes (SARA).

FIG. 12B is a plot graph showing a comparison between NNE predicted SARA combination and reference fluid density.

FIG. 13 is a plot graph showing an example of a mean and boundaries for a SARA reference fitting cluster in a fluid database.

FIG. 14 is a flowchart of an illustrative process for fluid analysis based on PSC data.

FIG. 15 is a block diagram of an illustrative computer system in which embodiments of the present disclosure may be implemented.

### DESCRIPTION OF ILLUSTRATIVE EMBODIMENTS

Embodiments of the present disclosure relate to downhole fluid analysis techniques for estimating fluid compositions and properties based on principal spectroscopy component (PSC) data. While the present disclosure is described herein with reference to illustrative embodiments for particular applications, it should be understood that embodiments are not limited thereto. Other embodiments are possible, and modifications can be made to the embodiments within the spirit and scope of the teachings herein and additional fields in which the embodiments would be of significant utility. Further, when a particular feature, structure, or characteristic is described in connection with an embodiment, it is submitted that it is within the knowledge of one skilled in the relevant art to implement such feature, structure, or characteristic in connection with other embodiments whether or not explicitly described.

It would also be apparent to one of skill in the relevant art that the embodiments, as described herein, can be implemented in many different embodiments of software, hardware, firmware, and/or the entities illustrated in the figures. Any actual software code with the specialized control of hardware to implement embodiments is not limiting of the detailed description. Thus, the operational behavior of embodiments will be described with the understanding that modifications and variations of the embodiments are possible, given the level of detail presented herein.

In the detailed description herein, references to “one embodiment,” “an embodiment.” “an example embodiment.” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used herein, the term “PSC” refers inclusively to both PSC and related multi-dimensional transformation techniques for producing PSC parameters or variables, where each resulting PSC variable is some combination of the original variables that is of reduced dimensionality. The term “spectroscopy” is used herein to refer to the multivariate nature of the data and not necessarily to a particular spectroscopy technique, e.g., optical spectroscopy or mass spectroscopy. For example, the original data from which PSC data is derived may include a combination of any of various sensor data channels for which a 10 degree of covariance exists. While the disclosed fluid analysis techniques will be described in reference to PSC data, it should be appreciated that embodiments of the present disclosure are not intended to be limited thereto and that the disclosed techniques may be applied to any data that has been transformed into a common multi-dimensional space.

As will be described in further detail below, the disclosed fluid analysis techniques may be used to estimate downhole fluid compositions and properties based on PSC data. The PSC data may be derived from measurements obtained from one or more downhole sensors. The downhole sensors may include optical sensors having a narrow-band filter, a broadband filter, a multivariate integrated computational element (ICE) core or any combination thereof. Such optical sensors may be coupled to a downhole tool disposed within a wellbore drilled into a subsurface hydrocarbon-bearing formation. In one or more embodiments, raw measurements collected by the optical sensors during a downhole operation along different sections of the wellbore may be transformed into PSC data and then provided as inputs to a fluid analysis model for performing real-time fluid analysis. For example, the fluid analysis model may be used to estimate fluid compositions and properties in real time as the downhole operation is performed along each section of the wellbore. In contrast with conventional sensor-based fluid analysis techniques, the disclosed PSC based fluid analysis techniques enable the inputs of the fluid analysis model to be independent of the particular type of sensors used or original sensor measurements acquired during the downhole operation. Accordingly, the disclosed techniques may be used to provide a universal, sensor-independent downhole fluid analyzer.

The disclosed techniques used to estimate fluid compositions and properties from PSC data inputs may include simulating optical sensor responses and providing enhanced ICE to PSC data transformation with merged sensor measurement and simulation inputs to overcome the limitations associated with conventional sensor-based calibration techniques and improve optical data transformation. In one or more embodiments, any uncertainty in the transformed PSC data and/or fluid composition and property predictions may be estimated using reference fluid type and composition/property supporting clusters. In some implementations, a neural network ensemble may be used to evaluate answer product prediction. Advantages of the disclosed techniques include, but are not limited to, improving optical sensor calibration procedures and ruggedizing downhole fluid identification and characterization. It can also benefit real-time prediction diagnostic and remedial analysis such as inconsistency detection, unknown fluid identification, and adaptive model reselection.

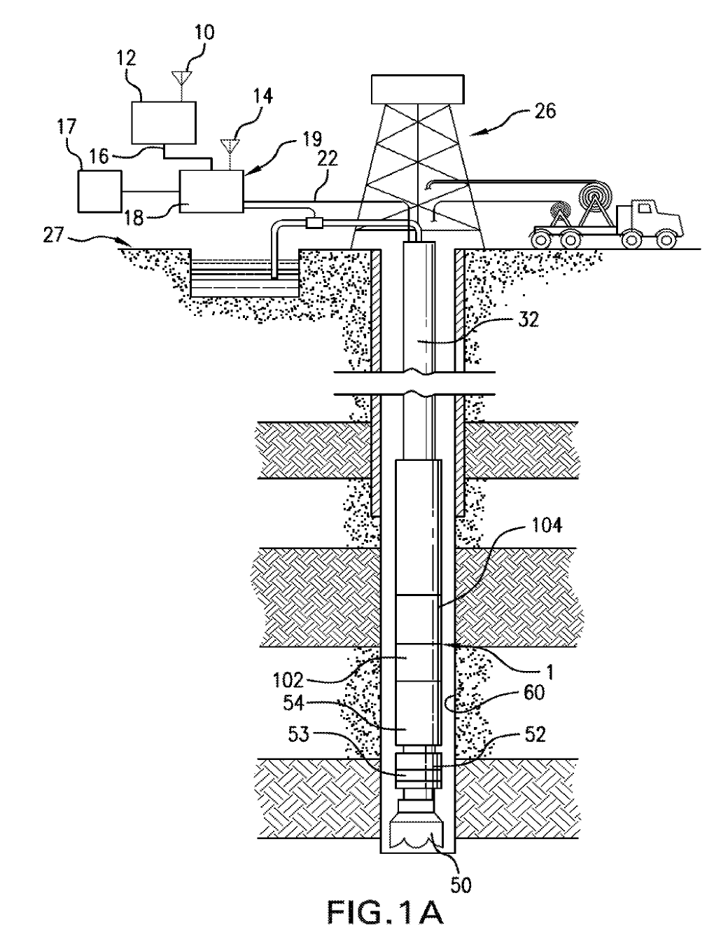

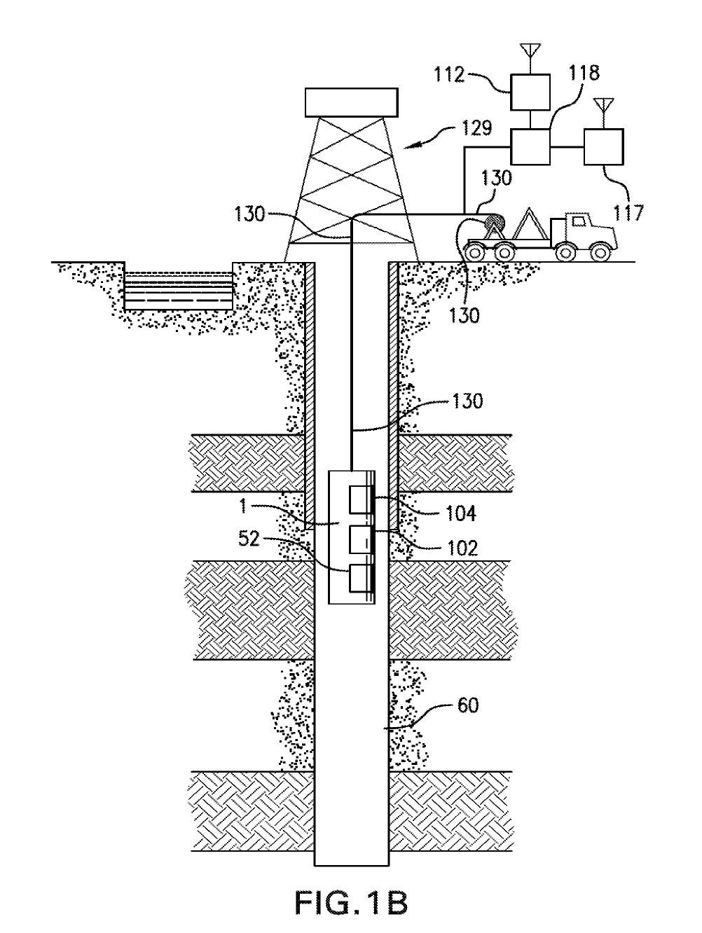

Illustrative embodiments and related methodologies of the present disclosure are described below in reference to FIGS. 1-15 as they might be employed, for example, in a downhole tool for performing real-time fluid analysis based on sensor measurements acquired along a wellbore during a downhole operation. Such a downhole fluid analyzer may be coupled to a work string disposed within the wellbore, e.g., as part of a bottom hole assembly coupled to a distal end of the work string within the wellbore. The downhole fluid analyzer tool may also include at least one processor and a memory for storing processor-readable instructions and data, as will be described in further detail below. Other features and advantages of the disclosed embodiments will be or will become apparent to one of ordinary skill in the art upon examination of the following figures and detailed description. It is intended that all such additional features and advantages be included within the scope of the disclosed embodiments. Further, the illustrated figures are only exemplary and are not intended to assert or imply any limitation with regard to the environment, architecture, design, or process in which different embodiments may be implemented.

FIG. 1A is a diagram of an illustrative well system for downhole fluid classification using PSC data. As shown in FIG. 1A, the well system includes a drill string 32 that extends from a drilling rig 26 into a wellbore 60 within a subsurface hydrocarbon bearing formation. Drilling rig 26 may include equipment for raising and lowering casing, drill pipe, coiled tubing, production tubing, other types of pipe or tubing strings or other types of conveyance vehicles, e.g., wireline or slickline, for performing various downhole operations at the wellsite. Such operations may include, but are not limited to, drilling, production, and stimulation operations performed along different sections of wellbore 60. Also, as shown in FIG. 1A, drill string 32 includes a drill bit 50, one or more optical sensors 52 coupled to a downhole tool 100. In one or more embodiments, downhole tool 100 may be a pump-out formation tester including a pump assembly to pump fluid samples out of the formation for testing and analysis. While not shown in FIG. 1A, it should be appreciated that drill string 32 may include other types of sensors in addition to optical sensors 52. Examples of such other sensors include, but are not limited to, density sensors for measuring fluid density and location sensors for determining the relative location or to position, direction, and azimuthal orientation of drill bit 50 or drill string 32 within wellbore 60 and the subsurface formation during downhole operations.

In one or more embodiments, optical sensors 52 and other sensors of drill string 32 may be used to detect and measure formation characteristics along wellbore 60 at any desired depth or location within the subsurface formation. For example, optical sensors 52 and other sensors may be used to measure fluid characteristics near drill bit 50 as well as other environmental parameters within the formation surrounding drill bit 50. Such environmental parameters may include, but are not limited to, pressure, temperature, and volume of fluids. However, it should be appreciated that the disclosed embodiments are not intended to be limited thereto and that, while optical sensors 52 are shown above drill bit 50 in FIG. 1A, optical sensors 52 may be positioned at any location along drill string 32. For example, in some implementations, optical sensors 52 along the internal or external surfaces of downhole tool 100.

In one or more embodiments, optical sensors 52 may include one or more integrated computational element (ICE) cores for detecting particular chemical compositions or properties of formation fluids. Such an ICE core may use electromagnetic radiation emitted from a light source to optically interact with a sample of fluid to determine one or more sample characteristics of the fluid. The sample may be of a multiphase wellbore fluid (comprising oil, gas, water, and solids, for example), for which a variety of fluid properties may be detected. Examples of such properties include, but are not limited to, C1-C5 hydrocarbon concentration, gas/oil ratio (GOR), SARA (saturates, aromatics, resins, and asphaltenes) concentration, CO2, H2O, synthetic drilling fluid (SDF) concentration, and specific gravity.

In one or more embodiments, measurements collected by optical sensors 52 and other sensors for a current section of wellbore 60 within the subsurface formation may be used by downhole tool 100 for real-time processing and qualitative and/or quantitative fluid analysis downhole. The current section of wellbore 60 may correspond to, for example, a current position of drill bit 50 within wellbore 60, e.g., as determined using location sensor measurements. As will be described in further detail below, the results of the fluid analysis performed by downhole tool 100 may be used to make real-time operational decisions related to the downhole operation being performed, e.g., making adjustments to a current path of wellbore 60 through the subsurface formation during a chilling operation. However, it should be appreciated that the techniques disclosed herein are not intended to be limited to drilling operations and that these techniques may be applied to other types of downhole operations.

As shown in the example of FIG. 1A, downhole tool 100 includes a data converter 102, a fluid analyzer 104, and a control unit 106. In one or more embodiments, data converter 102 may convert or transform the measurements obtained from optical sensors 52 to PSC data for use by fluid analyzer 104. For example, data converter 102 may transform the raw optical signals of one or more ICE cores of optical sensors 52 from a sensor parameter space associated with optical sensors 52 to a PSC parameter space associated with fluid classifier 104. The PSC data resulting from the transformed ICE data may then be provided as inputs to fluid analyzer 104. Measurements from other non-optical sensors, e.g., fluid density data from density sensors coupled to drill string 32, may be provided as additional inputs directly to fluid analyzer 104, without any conversion or transformation by data converter 102. In one or more embodiments, data converter 102 may be a neural network converter that uses one or more neural networks to perform the data transformation. As will be described in further detail below, such a neural network converter may use, for example, a neural network ensemble created from a plurality of neural networks, which have been combined to produce a desired output.

In one or more embodiments, fluid analyzer 104 may use the PSC data from data converter 102 to estimate fluid compositions and properties within the subsurface formation surrounding the current section of wellbore 60. As will be described in further detail below, the PSC data transformation performed by data converter 102 allows the inputs provided to fluid analyzer 104 from different types of sensors to be standardized regardless of the particular sensor configuration or element design. This in turn may allow fluid analyzer 104 to be used as a universal fluid analyzer that operates with generalized or generic inputs, which are independent of the type of sensor or original sensor data from which they are derived.

In one or more embodiments, control unit 106 of downhole tool 100 may use additional information obtained from a fluid type classifier (not shown), or fluid classification model thereof to refine the output of fluid analyzer 104 for the current and/or subsequent sections of wellbore 60. In one or more embodiments, such information may be used along with the estimated fluid compositions and properties to refine a fluid analysis model, as will be described in further detail below. Control unit 106 may include, for example, a signal processor (not shown), a communications interface (not shown) and other circuitry necessary to achieve the objectives of the present disclosure, as would be understood by those of ordinary skill in the relevant art having the benefit of this disclosure. In one or more embodiments, control unit 106 may use the signal processor to send control signals via the communications interface to other components of drill string 32 for purposes of controlling or making appropriate adjustments to the downhole operation being performed. The particular adjustments may be based on the fluid types identified by fluid analyzer 104 for the current section of wellbore 60. For example, such control signals may be used to control or adjust the path of wellbore 60 for subsequent wellbore sections to be drilled within the subsurface formation during a drilling operation. Control unit 106 in this example may send appropriate control signals to a downhole motor assembly (not shown) for purposes of controlling the direction or orientation of drill bit 50 according to the adjusted path of wellbore 60. The adjusted path may be one that has been determined to be more optimal for hydrocarbon recovery based on the estimated fluid compositions and properties (or fluid profile) of the formation.

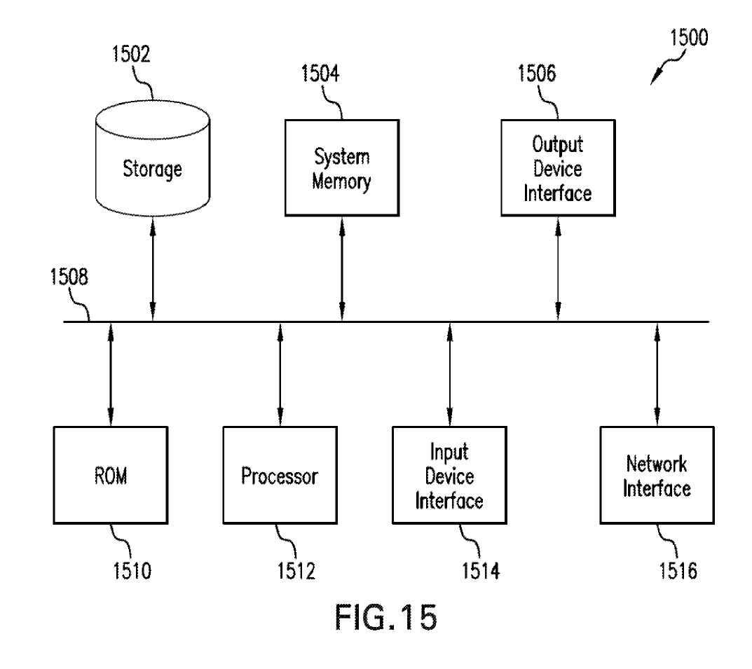

In one or more embodiments, control unit 106 may also transmit the fluid compositions and properties estimated by fluid analyzer 104 to a surface processing unit 19 located at a surface 27 of the wellsite. As shown in FIG. 1A, surface processing unit 19 may include a computing device 18 communicatively coupled to the components of drill string 32, including downhole tool 100 and optical sensors 52, via a communication path 22. Computing device 18 may store the fluid types in a local memory or data store 17. Additionally or alternatively, computing device 18 may send the fluid compositions and properties to another computing device of a data processing unit 12 via, for example, a wired connection 16 or a wireless connection established between transceivers 14 and 10 of surface processing units 19 and 12, respectively. Data processing unit 12 may be, for example, a remote data storage or database system including a database server that is communicatively coupled to computing device 18 via a communication network. Such a communication network may be, for example, a local-area network, a medium-area network or a wide-area network, e.g., the Internet. The computing devices of data processing units 12 and 19 may be implemented using any type of computing device, an example of which will be described in further detail below with respect to FIG. 15.

Although only data converter 102, fluid analyzer 104, and control unit 106 are shown in FIG. 1A, it should be appreciated that downhole tool 100 may include additional components, modules, and/or sub-components as desired for a particular implementation. It should also be appreciated that data converter 102, fluid analyzer 104, and control unit 106 may be implemented in software, firmware, hardware, or any combination thereof. Furthermore, it should be appreciated that embodiments of data converter 102, fluid analyzer 104, and control unit 106, or portions thereof, can be implemented to nm on any type of processing device including, but not limited to, a computer, workstation, embedded system, networked device, mobile device, or other type of processor or computer system capable of carrying out the functionality described herein.

In some implementations, the data transformation and fluid analysis functions performed by data converter 102 and fluid analyzer 104, respectively, as well as the control functions performed by control unit 106 of downhole tool 100, as described above, may be performed by computing device 18 at the surface. For example, the downhole measurements collected by optical sensors 52 and other sensors of drill string 32 may be transmitted to computing device 18 via communication path 22. In some cases, optical sensors 52 may include a signal processing apparatus for transmitting the downhole measurements as signals directly to computing device 18 via communication path 22 along drill string 32.

As described above, embodiments of the present disclosure may be applied to any of various downhole operations. Thus, while the example in FIG. 1A is described above in the context of drill string 32 or a drilling assembly, it should be appreciated that the disclosed embodiments may be implemented using other types of downhole assemblies or tubular strings. For example, optical sensors 52 and downhole tool 100 may be deployed within wellbore 60 as part of a wireline assembly, as will be described in further detail below with respect to FIG. 1B.

FIG. 1B is a diagram of an illustrative wireline system for a PSC based downhole fluid classifier. As illustrated in FIG. 1B, downhole tool 100 may be employed with “wireline” systems in order to carry out logging or other operations. For example, instead of using the drill string 32 of FIG. 1A to deploy downhole tool 100 within wellbore 60, downhole tool 100 may be lowered into wellbore 60 by a wireline conveyance 130, as shown in FIG. 1B. As in FIG. 1A, downhole tool 100 includes a data converter 102 and fluid classifier 104. Downhole tool 100 as shown in FIG. 1B also incorporates optical sensors 52 and other non-optical sensors (not shown).

Conveyance 130 as shown in FIG. 1B can be anchored in the drill rig 129 or portable means such as a truck. Conveyance 130 can be one or more wires (e.g., a wireline), slickline, cables, or the like, as well as tubular conveyances such as coiled tubing, joint tubing, or other tubulars. Conveyance 130 may provide support for downhole tool 100 and also, enable communications between the tool and a surface processing unit 118 at the surface. In the example of FIG. 1B, a computing device of surface processing unit 118 may perform the functions performed by control unit 106 of downhole tool 100 as described above with respect to FIG. 1A. Conveyance 130 may include fiber optic cabling for carrying out communications. Additionally, power may be supplied via conveyance 130 to meet power requirements of downhole tool 100 and its components. For slickline or other tubing configurations, power can be supplied downhole with a battery or via a downhole generator.

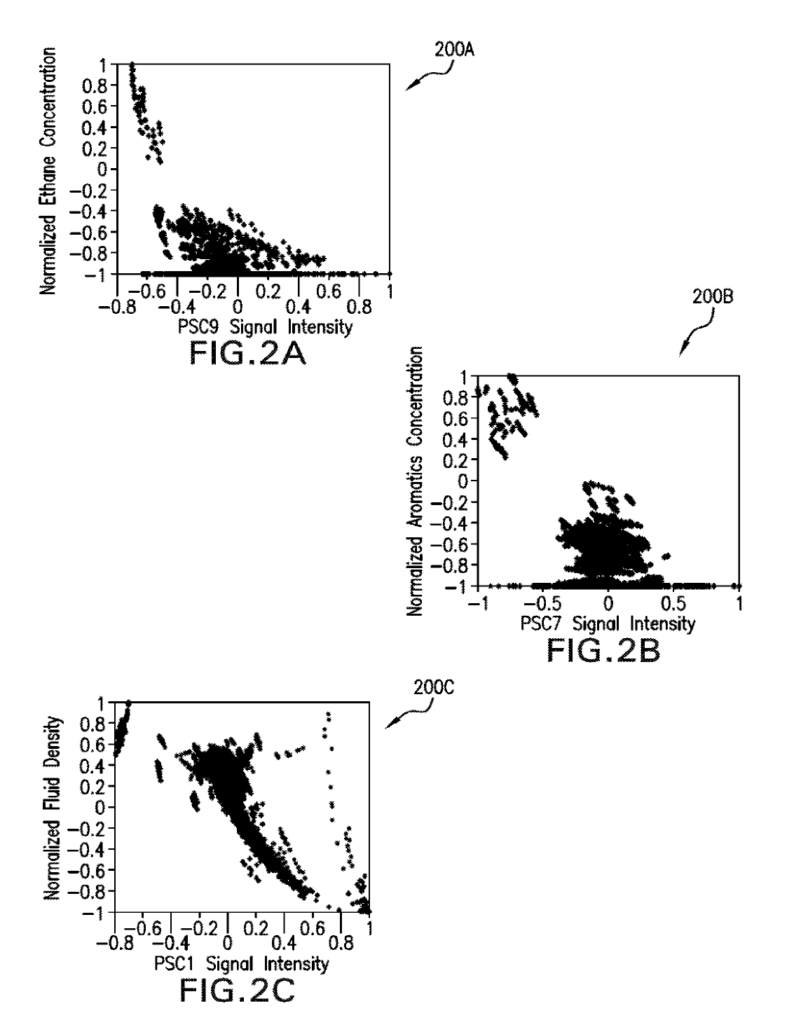

It should be noted that while FIGS. 1A and 1B generally depict a land-based or onshore operation, the techniques described herein are also applicable to offshore operations that employ floating or sea-based platforms and rigs, without departing from the scope of the disclosure. Also, while FIGS. 1A and 1B depict vertical wellbores, the present disclosure is equally well-suited for use in wellbores having other orientations, including horizontal wellbores, slanted wellbores, multilateral wellbores or the like. Further, while wellbore 60 is depicted in FIGS. 1A and 1B as a cased hole, it should be appreciated that downhole tool 100 may be equally well suited for use in open hole operations. Additional details relating to the real-time data processing and fluid analysis techniques disclosed herein. e.g., as performed by downhole tool 100 of FIGS. 1A and 1B, will now be described using FIGS. 2-15.

Fluid optical spectroscopy based modeling is fundamental to downhole optical fluid analysis. However, optical spectroscopic data are rarely used in full wavelength range because of its very high dimension. In one or more embodiments, PSC data may be generated by applying any of various principal component analysis (PCA) techniques to high-dimension pressure, volume and temperature (PVT) spectroscopic data. Such data may include laboratory measurements of fluid spectroscopies over a full or selected wavelength range or ranges, e.g., as obtained using one or more high-resolution laboratory spectrometers. The spectroscopy data for different types of fluids may also be stored in an optical-PVT database for later access and retrieval. In one or more embodiments, the generated PSC data may be provided as inputs to a downhole fluid analyzer, e.g., fluid analyzer 104 of FIGS. 1A and 1B, as described above. While there may be a significant reduction in dimensionality associated with such PSC input data relative to the PVT data from which it was derived, the PSC data may still adequately capture the most relevant features of the original full-range fluid spectroscopy information. As such, even just a few PSC inputs may be sufficient to estimate a variety of different fluid compositions and properties for purposes of multivariate calibration of the downhole fluid analyzer (or fluid analysis model thereof), as will be described in further detail below.

FIGS. 2A-2C are plot graphs of illustrative data relationships between PSC signal intensity and different fluid properties. The PSC signal intensity in each plot graph may represent one of a plurality of PSC inputs applied to the downhole fluid analyzer described above for estimating the fluid properties. For purposes of this example, it is assumed that there are a total of nine PSC inputs. However, embodiments are not intended to be limited thereto and any number of PSC inputs may be used as desired for a particular implementation. It is also assumed that the spectroscopy data in this example is within a normalized data range from −1 to +1 for both PSC inputs and the estimated fluid properties. It is further assumed that the PSC data represented in these plot graphs was derived from optical transmittance spectroscopies (e.g., approximately 3400 spectra) of more than 200 samples of various types of fluids with wavelengths ranging from 1200 to 3350 nanometers. Examples of such fluid types include, but are not limited to, heavy oil, medium oil, light oil, condensates and hydrocarbon gas, water, nitrogen, and CO2 samples with different compositions and properties. Such spectroscopy data may include laboratory measurements of the fluid samples within a given temperature and pressure range for various types of fluids. The measured temperature range associated with the fluid samples in this example is assumed to be from 150 to 250 Fahrenheit. The pressure range for the fluid samples is assumed to be from 3000 to 12000 pounds per square inch (psi). The laboratory measurements may be stored in an optical-PVT database, as described above. In one or more embodiments, the stored measurements for selected fluids of interest may be retrieved from the optical-PVT database and converted or transformed into PSC data using any of various principal component analysis (PCA) techniques.

Thus, FIG. 2A is a cross-plot 200A of a ninth PSC input (PSC9) of the downhole fluid analyzer in this example relative to ethane gas concentration. FIG. 2B is a cross-plot 200B of a seventh PSC input (PSC7) relative to aromatic concentration. FIG. 2C is a cross-plot 200C of a first PSC input (PSC1) versus fluid density. However, it should be appreciated that the disclosed fluid analysis techniques may be applied to other types of fluid compositions or properties and PSC inputs.

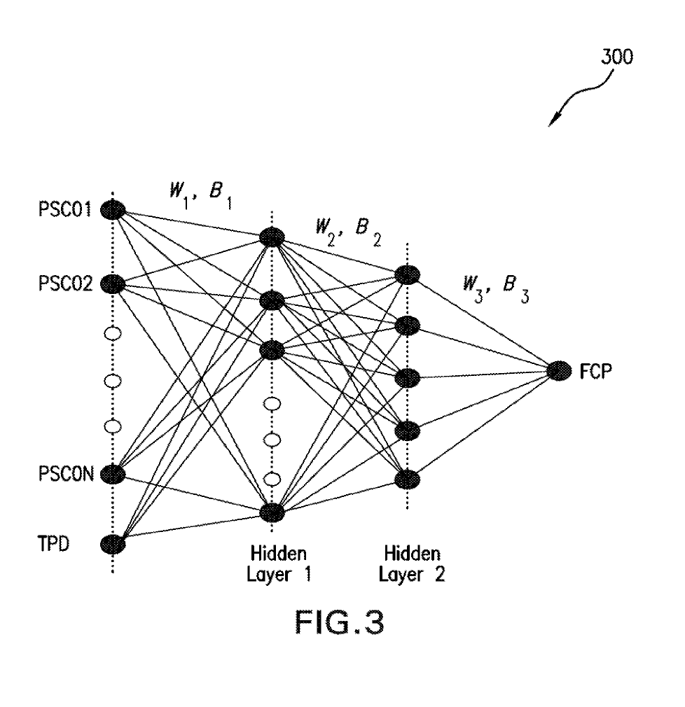

In one or more embodiments, the downhole fluid analyzer may use a fluid analysis model in the form of a neural network with a nonlinear multi-input/single-output (MISO) structure for estimating each fluid composition or property (FCP) of interest based on the PSC input data. FIG. 3 shows an example of such a MISO structure for a neural network 300 having multiple layers of neural network nodes (or “neurons”). For example, neural network 300 may be used to estimate at least one of ethane gas concentration, aromatic concentration, or fluid density, as represented in each of cross-plots 200A, 200B, and 200C of FIGS. 2A, 2B, and 2C, respectively. The layers of neural network 300 may include an input layer, a plurality of hidden layers, and an FCP output layer. The input layer of neural network 300 may include separate nodes or neurons for any number (N) of PSC inputs (e.g., from PSC01 to PSC0N) and one or more temperature, pressure and density (TPD) inputs. However, it should be appreciated that embodiments of the present disclosure are not intended to be limited to the particular neural network structure shown in FIG. 3 and that any of various neural network structures may be used as desired for a particular implementation. Also, while neural network 300 is shown in FIG. 3 with two hidden layers, it should be appreciated that any number of hidden layers may be used as desired for a particular implementation.

In one or more embodiments, neural network 300 may use a nonlinear transfer function to define the output of each hidden neuron of the hidden layers based on one or more of the PSC and other inputs from the input layer. The nonlinear transfer function may be, for example, a hyperbolic tangent sigmoid function. Additionally, neural network 300 may use a linear transfer function to define the output of each output neuron of the output layer based on the outputs of the hidden neurons. The outputs of the output neurons may then be used to estimate the FCP, as shown in FIG. 3. The neuron output for the first hidden layer, the second hidden layer, and the output layer of neural network 300 may be calculated using, for example, Equations (1), (2), and (3), respectively:

$$\begin{matrix}{a}_{1} = {f}_{1} ( {n}_{1} ) = \frac{{e}^{{n}_{1}} - {e}^{- {n}_{1}}}{{e}^{{n}_{1}} + {e}^{- {n}_{1}}} , {n}_{1} = {W}_{1} \times P + {B}_{1} & ( 1 ) \\ {a}_{2} = {f}_{2} ( {n}_{2} ) = \frac{{e}^{{n}_{1}} - {e}^{- {n}_{1}}}{{e}^{{n }_{2}} + {e}^{- {n}_{2}}} , {n}_{2} = {W}_{2} \times {a}_{1} + {B}_{2} & ( 2 ) \\ {a}_{3} = {n}_{3} = {W}_{3} \times {a}_{2} + {B}_{3} & ( 3 )\end{matrix}$$

where: aₜ is the output of the first hidden layer, a₂ is the output of the second hidden layer, and a₃ is the output of the neural network; n₁, n₂, and n₃ are neuron inputs to the respective hidden and output layers; and W₁, B₁, W₂, B₂, and W₃, B₃ are parameters representing pairs of connection coefficients for neural network 300, which may be optimized through training during a calibration of neural network 300.

In one or more embodiments, neural network 300 may include multiple neural networks in the form of a neural network ensemble (NNE), where each member neural network may be configured with a different number of hidden neurons within one or more hidden layers or calibrated with a different number of inputs or input neurons for the input layer. For example, a variable number of candidate PSC inputs may be used to calibrate neural network 300 for estimating different fluid compositions and properties. In Equation (1) above, P may represent a selected set of candidate PSC inputs indexed from one to any integer number N (e.g., ranging from PSC01 to PSC0N). The selected set may be one of a plurality of different candidate PSC input sets.

In one or more embodiments, the calibration of neural network 300 may be performed using machine learning to minimize the difference between the predicted output of neural network 300 (or NNE thereof) and a training target assigned to each particular FCP for a given set of candidate PSC inputs. The determination of optical inputs for each FCP prediction may be optimized through machine learning during calibration. Such optimization may include, for example, performing backward stepwise input selection to obtain an optimized parameter combination for each set of PSC candidate inputs and an evaluation of outputs to minimize calibration error.

In one or more embodiments, the output of neural network 300 may represent an average of the estimates or FCP predictions output by a plurality of member networks within an NNE of neural network 300, where each member network may be trained or calibrated using a different subset of PSC candidate inputs or subset of PSC inputs. The PSC inputs in this example may be derived from a full or selected range of fluid transmittance spectroscopy measurements retrieved from a database of optical PVT (pressure, volume, and temperature) laboratory data, as described above. In some implementations, such an optical-PVT database may include PSC data generated from applying any of various principal component analysis (PCA) techniques to laboratory measurements of fluid spectroscopies over a full or selected wavelength range or ranges.

Additionally or alternatively, the PSC data for different types of fluids may be stored in a PSC database accessible to neural network 300 for purposes of calibration. For example, neural network 300 may access such a database to retrieve PSC data for a selected number of reference fluids that may be representative of the types of fluids expected within the formation. Examples of such formation fluids include, but are not limited to, oils, water, nitrogen gas, hydrocarbon gas and condensates. The selected reference fluids may also include some non-reservoir fluids including, for example and without limitation, toluene, dodecane and pentanediol. The addition of such non-reservoir reference fluids may be used to improve the variety of downhole fluid patterns represented by the PSC input data and thereby, fill any gaps in the calibration data range.

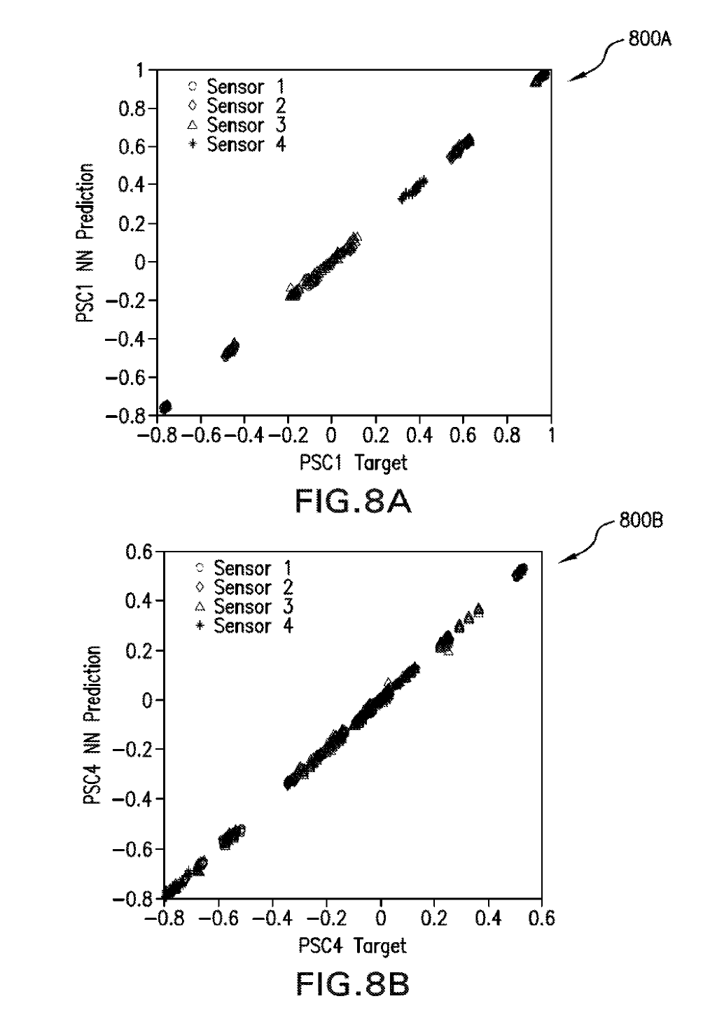

As described above, the PSC input data applied to neural network 300 for performing the fluid analysis techniques disclosed herein may be derived from fluid measurements obtained from one or more downhole sensors (e.g., optical sensors 52 of FIG. 1A or 1B, as described above). In one or more embodiments, a reverse transformation model may be used to transform such measurements from a sensor parameter space to a multi-dimensional PSC parameter space. An example of such a reverse transformation model is shown in FIG. 7, as will be described in further detail below. To make the reverse transformation more robust, the model may be calibrated using a combination of sensor and PSC data pairs. To ensure that such calibration data adequately represents diverse fluid types over a wide dynamic range of fluid compositions or properties, the sensor data should reflect measurements for a sufficiently large number of reference fluids. Thus, in order to avoid limiting the calibration data to only a small or insufficient number of reference fluids, the available sensor data may be expanded to include synthetic sensor data. The synthetic sensor data may represent simulated sensor responses generated using a forward transformation model. In one or more embodiments, the forward transformation model may be a neural network having a MISO structure similar to that of neural network 300.

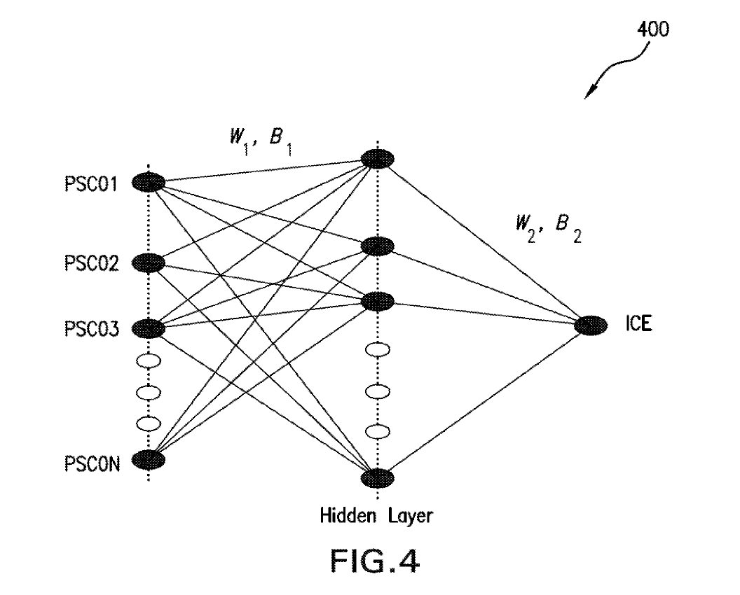

FIG. 4 is a diagram of an illustrative forward transformation model 400 with such a MISO neural network structure. As shown in the example of FIG. 4, forward transformation model 400 may be used to simulate an optical sensor response for a given number of PSC inputs (from PSC01 to PSC0N). The simulation using forward transformation model 400 may involve, for example, transforming the PSC input data to a synthetic or virtual parameter space associated with an ICE core of an optical sensor associated with a particular downhole fluid analysis tool (e.g., downhole tool 100 of FIG. 1A or 1B, as described above). To optimize the optical sensor data transformation, forward transformation model 400 may be calibrated for an initial set of reference fluids. Simulation data for additional fluids may then be generated using the calibrated forward transformation model.

As shown in FIG. 4, forward transformation model 400 may include a single hidden layer for transforming calibration inputs to a calibration output for each ICE core or sensing element of the optical sensor. The calibration inputs in this example may include a set of multivariate candidate PSC input parameters. It should be noted that the number of candidate PSC parameters used to calibrate forward transformation model 400 in FIG. 4 may vary from the number of candidate PSC input parameters selected for calibrating neural network 300 in FIG. 3, as described above, depending on, for example, the desired level of accuracy for each calibration and associated input and output data relationships. In some implementations, forward transformation model 400 may use nonlinear and linear transfer functions for the respective hidden and output layers that are the same or similar to those used by neural network 300 for FCP estimation, as described above. However, only Equations (1) and (3) above may be needed to determine the neural network output for the single hidden layer of forward transformation model 400 in the example of FIG. 4.

In one or more embodiments, forward transformation model 400 may be used to simulate optical sensor responses for additional fluids that were not included in the initial set of reference fluids. The simulated sensor responses may be combined with measured optical responses to the reference fluids from a laboratory spectrometer for a more robust 1o calibration of the reverse transformation model. Because the wavelength range used in calculating PSC is usually wider than operating wavelength range for each particular sensor element, the calibrated forward MISO transformation algorithm can be used as synthesizer to generate optical response of each sensing element from the orthogonal PSC data inputs. To improve the results of forward transformation, the reference fluids selected for calibration may include fluids that are representative of the primary fluid types of interest. Examples of such fluid types may include, but are not limited to, dead oil, medium and light oil, condensate and gas, and water and nitrogen.

Once forward transformation model 400 has been calibrated, it may be used to map most, if not all, of the fluid spectroscopy data stored in the optical-PVT and/or PSC database from PSC parameter space to sensor parameter space. The quality of the data mapping of the PSC data to the expected optical sensor responses for the various fluids represented in the database may be evaluated through clustering and boundary analysis. For example, the fluids represented in the PSC database may be normalized and grouped into multiple clusters through hierarchical clustering analysis for fluid type classification. The measured optical sensor responses for a sample of fluid selected as a reference fluid is expected to be similar to the simulated optical sensor responses for other fluids that belong to the same PSC cluster for a fluid type corresponding to the reference fluid sample.

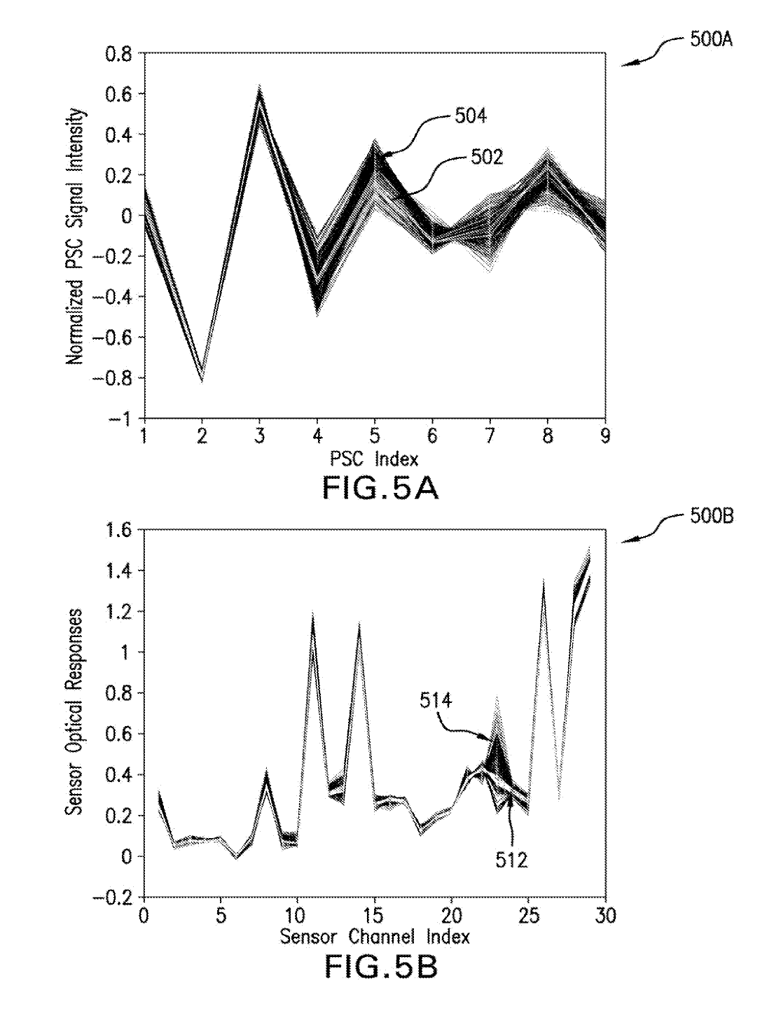

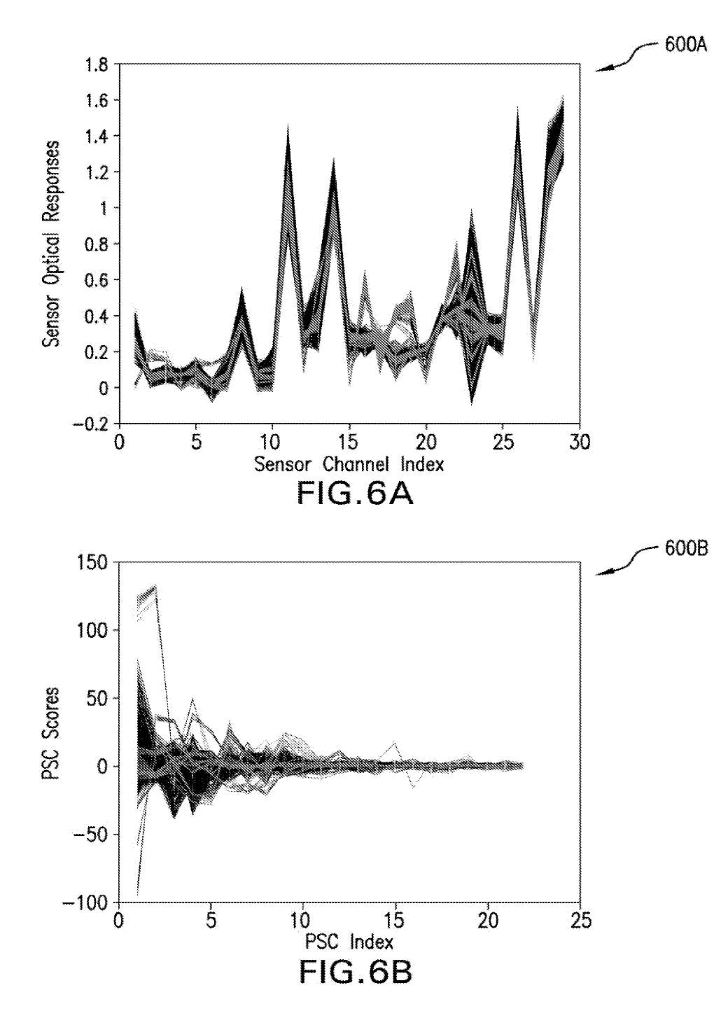

FIG. 5A is a plot graph 500A showing an example of PSC data for a cluster of fluids in which a downhole fluid sample (e.g., an oil sample) under multiple measurement conditions may be selected as reference fluid. For purposes of this example, it may be assumed that there are approximately 40 other fluid samples measured with different temperatures and pressures within the same PSC cluster as the reference fluid sample. It may also be assumed that the PSC data for the reference fluid sample in this example corresponds to portion 502 of plot graph 500A and portion 504 corresponds to the PSC data for the other fluid samples.

FIG. 5B is a plot graph 500B showing measured optical responses 512 and simulated optical responses 514 to the fluids of FIG. 5A in sensor parameter space. For purposes of this example, it may be assumed that the sensor has at least 29 sensing elements or channels. The measured optical responses may be collected for the same reference fluid, and the simulated optical responses may be applied to the other samples in the cluster of FIG. 5A using, for example, forward transformation model 400 of FIG. 4, as described above. The simulated optical sensor responses may be validated using additional testing data on fluids measured in actual sensor parameter space. However, if such validation fluids are not available, it may be possible to make a reasonable engineering assessment as to the quality of the data mapping between the actual/measured and simulated sensor responses by comparing the results depicted in FIGS. 5A and 5B. In some implementations, a boundary criterion may be used to remove certain fluid samples from the transformed data set if the simulated sensor responses are determined to be outside of a calibration data range associated with the reference fluids. The boundary criterion in this context may represent a kind of error tolerance threshold for evaluating the forward transformation.

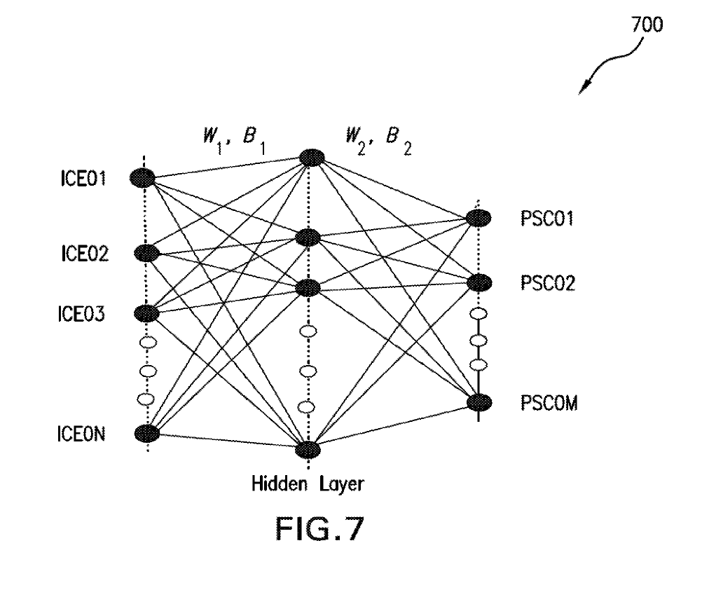

In one or more embodiments, the simulated sensor data associated with the identified fluid types or PSC clusters may be combined with the original data set of reference fluids in order to construct an extended data ensemble for calibrating the reverse transformation model. FIGS. 6A and 6B provide a comparison between calibration data of original reference fluids and extended fluids. FIG. 6A is a plot graph 600A showing an example of candidate calibration inputs. FIG. 6B is a plot graph 600B showing outputs with reference fluid data and extended data prior to normalization.

FIG. 7 is a diagram of an illustrative multi-input/multi-output (MIMO) neural network structure of a reverse transformation model 700. The calibration of reverse transformation model 700 may include applying sensor measurements and simulation data as training data that are similar to noisy inputs applied to machine learning. This may allow the resulting model to be more robust than training with clean inputs, as the noisy inputs may be more representative of actual downhole conditions that may be encountered during real-time data processing of optical sensor measurements collected along a wellbore within a subsurface formation.

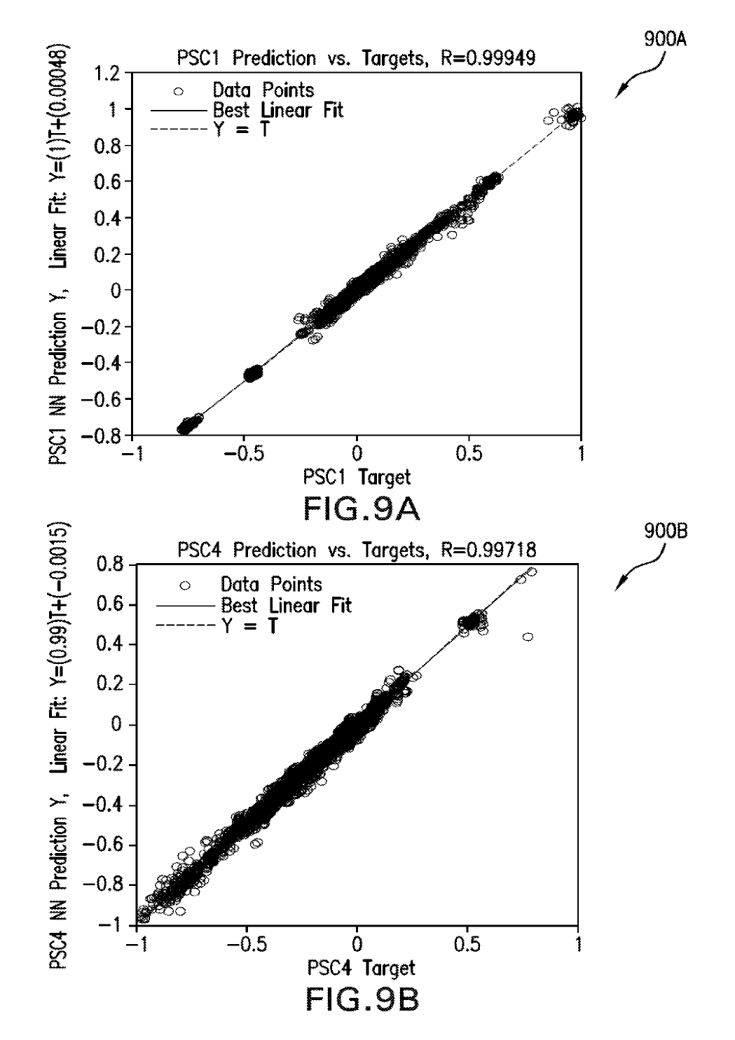

FIGS. 8A and 8B are cross-plots showing calibration results of reverse transformation on two outputs with four sensors by using reference fluids alone. FIG. 8A is a cross-plot 800A of a training target against a neural network (NN) prediction for a first set of PSC input data (PSC1). FIG. 8B is a cross-plot 800B of the training target against NN prediction for a fourth set of PSC input data (PSC4). The number of reference fluids used may vary (e.g., from 9 to 13) for different sensors. The distribution of the data as shown in cross-plots 800A and 800B may be sensor dependent and scattered with various gaps over the data range. A reverse transformation model trained on such sparse data may have the risk of over-fitting the training data and therefore, may not generalize well to actual data during downhole applications.

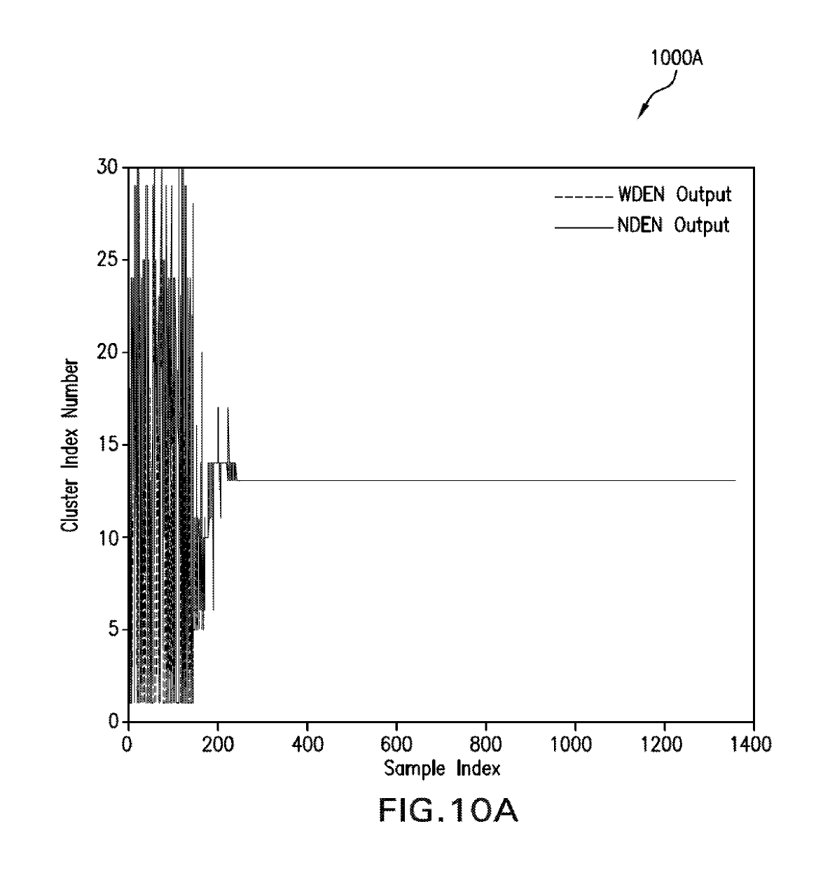

By contrast, cross-plots 900A and 900B of FIGS. 9A and 9B, respectively, present calibration results of the reverse transformation model for the same outputs of the reverse transformation of FIGS. 8A and 8B, except with a single sensor and extended data inputs. The extended data inputs may include measured sensor responses from a small number of reference fluids and simulated sensor responses from a large number of additional fluids. Unlike the outputs shown in FIGS. 8A and 8B, the data range for each output parameter shown in FIGS. 9A and 9B is improved for the particular fluid type of interest.

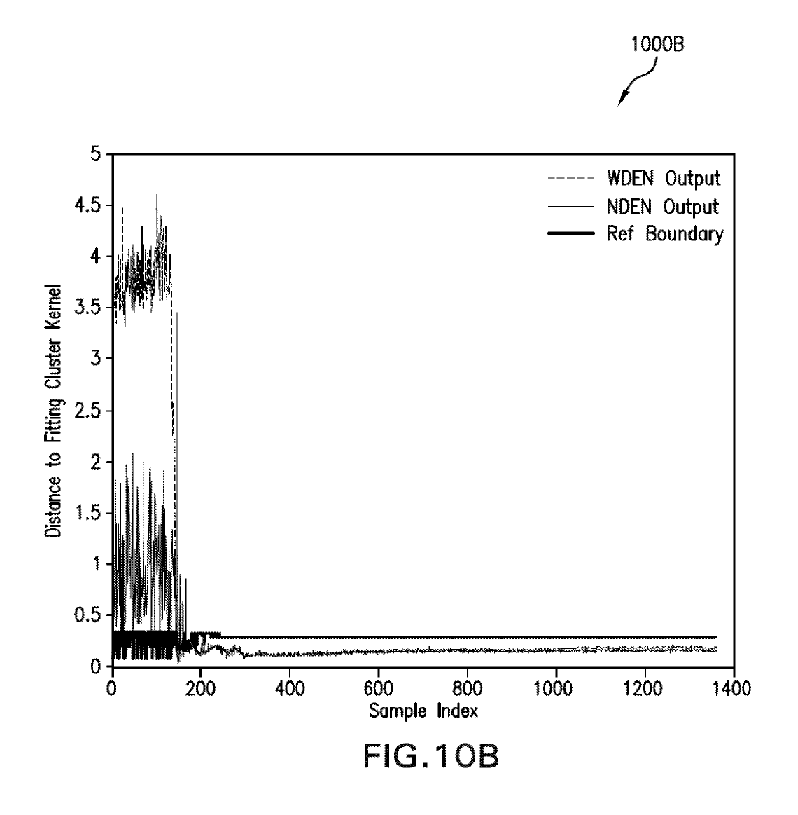

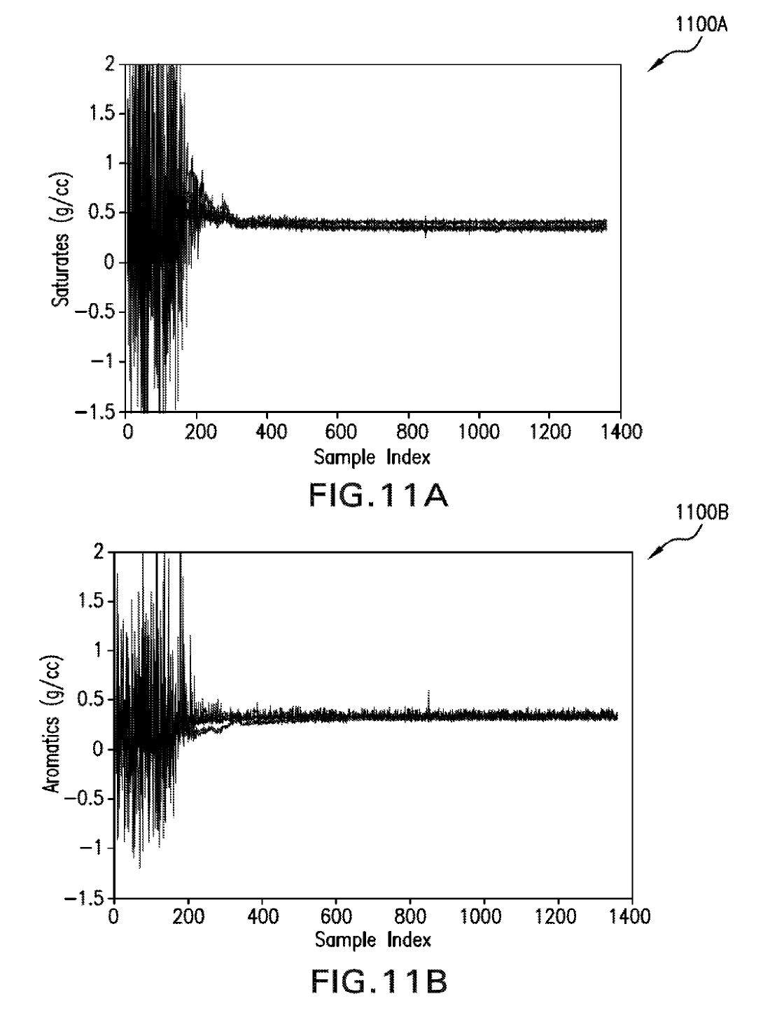

FIGS. 10A-13 will be used to describe an example of applying the disclosed techniques for estimating fluid compositions and properties in the context of real-time fluid analysis during a downhole operation performed along a wellbore within a subsurface formation. For example, optical sensor data from one or more sensors coupled to a downhole tool disposed within the wellbore may be processed using PSC data standardization techniques, e.g., by transforming the acquired sensor data from a sensor parameter space to a PSC parameter space, as described above. The processed (or transformed) data may then be applied as inputs to a predictive model for fluid characterization, e.g., by estimating fluid compositions and properties of the subsurface formation surrounding different sections of the wellbore as the downhole operation is performed.

In one or more embodiments, the real-time fluid analysis may be performed by a fluid analyzer of the downhole tool based on PSC input data derived from measurements obtained from the sensor(s) along a current section of the wellbore. The fluid analyzer may use a selected subset of the available PSC inputs and other sensor data (e.g., fluid density, bubble point and/or compressibility measurements) to perform fluid classification. In one or more embodiments, the fluid classification may be performed using cluster mean supporting vectors and nearest neighbor matching to identify one or more fluid types from the applied PSC data. For each cluster, the mean vector and sample standard deviation with respect to the sample-to-mean distance may be calculated and stored as fluid type supporting vectors. In one or more embodiments, the fluid type supporting vectors may be generated from with-density (WDEN) and without-density (NDEN) clustering analysis 1o based on PSC and other sensor data relating to diverse fluid samples. Such data may be applied as training data for calibrating a fluid analysis model used by the downhole fluid analyzer and may be stored in a standard oil library and/or other fluid database accessible to the downhole fluid analyzer for real-time data processing.

FIG. 10A is a plot graph 1000A showing examples of WDEN and NDEN PSC data that has been grouped into a fluid type cluster. The fluid type cluster in this example may have a particular cluster index number (e.g., 13), which corresponds to a pre-defined fluid pattern associated with the particular fluid type, e.g., heavy to medium oil, as identified for the cluster based on data obtained over a stabilized period of operation. FIG. 10B is a plot graph 1000B of fluid PSC data to cluster kernel distance, which is well within the reference boundary of the training data in the oil library or fluid database described above. The output of the WDEN and NDEN PSC data classification may be important for assessing any uncertainty in the data standardization. For example, the level of uncertainty with respect to the optical data transformation from the sensor parameter space to the PSC parameter space may be relatively low if the results of both WDEN and NDEN fluid classifications are found to be consistent, e.g., within a given error tolerance. The WDEN and NDEN classification outputs may also be used for density data assessment. For example, for the same PSC inputs, WDEN classification may produce a different output if additional density inputs from other sensors are inaccurate. The fluid classification in this example may be performed using, for example, a fluid classification model in the form of a neural network model including one or more neural networks for identifying different fluid types using supervised machine learning.

The fluid analysis model in this example may use relatively more PSC inputs than the number of inputs used for fluid classification. For a given number of inputs, an optimal combination of candidate inputs may be determined through calibration of the fluid analysis model. The calibrated fluid analysis model or portion thereof may then be used to estimate or predict a particular fluid composition or property (FCP). As described above, the fluid analysis model may be implemented as a neural network ensemble (NNE) with a plurality of member neural networks for estimating or predicting various fluid compositions and properties.

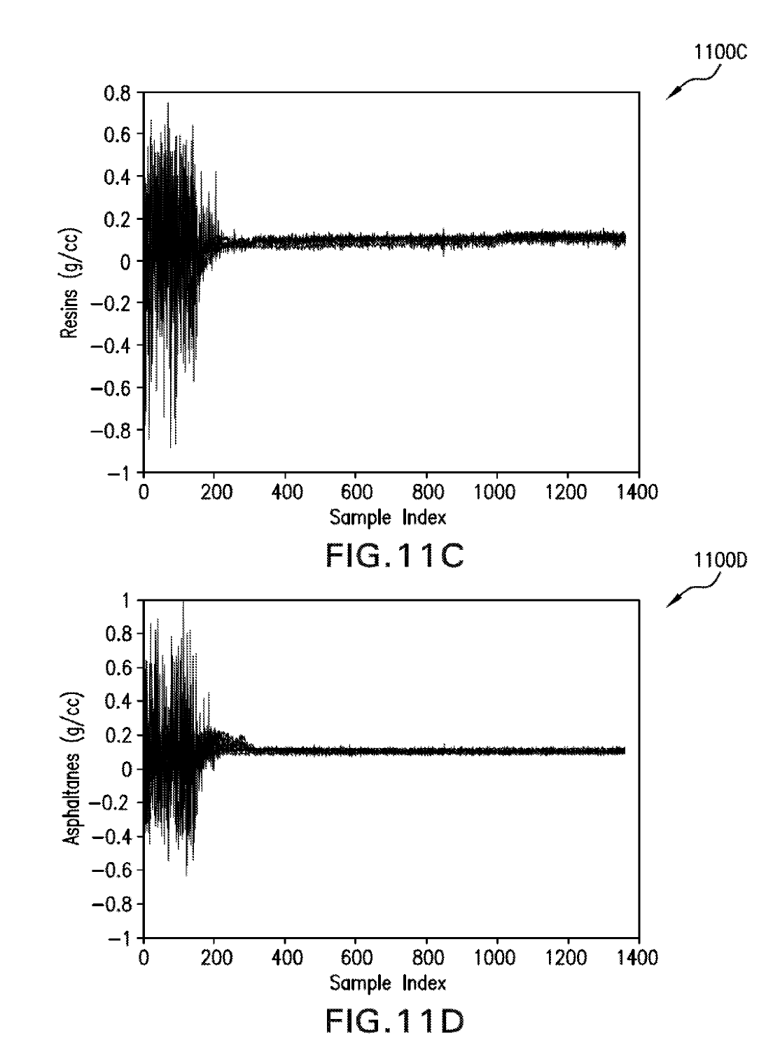

FIGS. 11A-11D are plot graphs of illustrative member network predictions for the concentration of saturates, aromatics, resins, and asphaltanes, respectively based on a variable number of inputs (e.g., ranging from 4 to 11 inputs) for the same field example. The member network predictions are generally well converged in this example, and the ensemble output may represent an arithmetic average with low uncertainty.

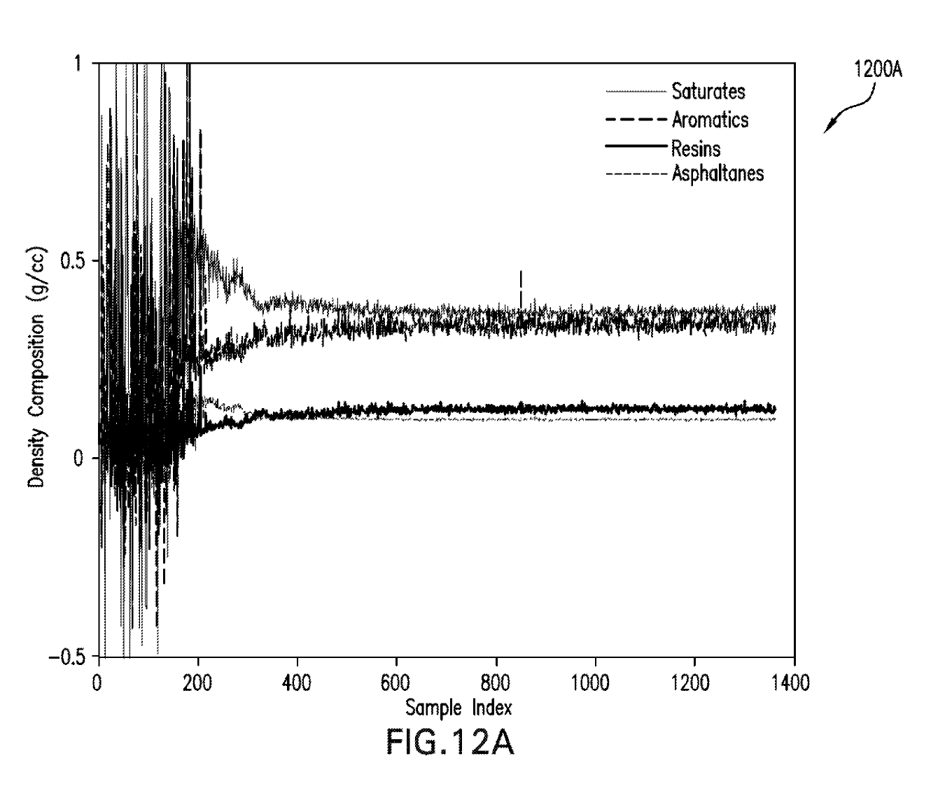

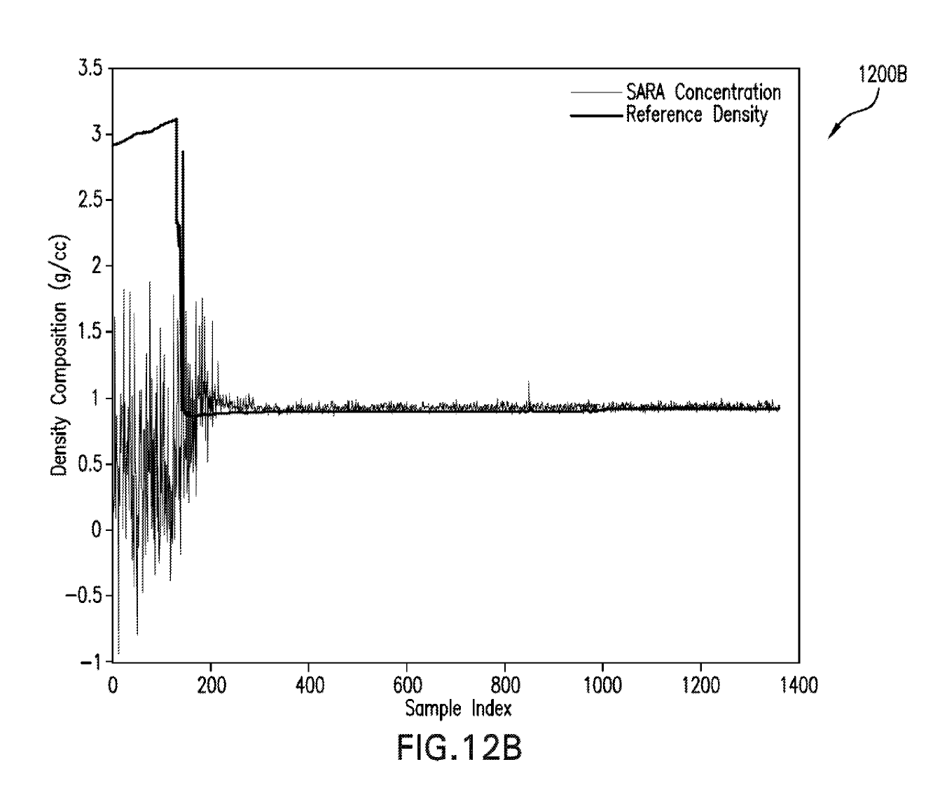

FIG. 12A is a plot graph 1200A of the NNE predictions on saturates, aromatics, resins, and asphaltanes. For heavy to medium oil in this example, the gas concentration may be very low, and the SARA (saturates, aromatics, resins and asphaltanes) combination may be a good estimate of fluid density. FIG. 12B is a plot graph 1200B showing a comparison between NNE predicted SARA combination and reference fluid density. The results are generally consistent over a stabilized portion of the plotted data range, which may indicate a low level of uncertainty in the resulting predictions.

The issue on NNE prediction may be indicated by: (a) any unmatched fluid types from WDEN and NDEN classification; (b) any unknown fluid PSC data to kernel cluster distances that exceed boundary criterion of training data; (c) a lack of convergence between the predictions by different member networks; and (d) inconsistent predictions from fluid compositional estimates evaluated using a mass balance equation. If any uncertainty issues are detected with respect to the FCP predictions produced by the NNE during real time data processing, the predictions made by individual member networks prediction may be compared to reference or training data compositions in fluid type supporting vectors/matrices to determine how to adjust or refine the NNE to better fit the training data.

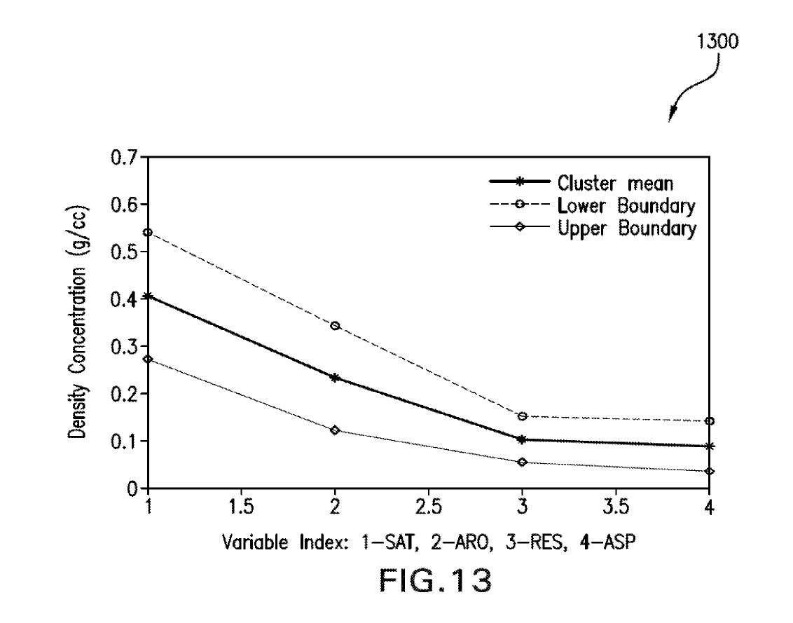

FIG. 13 is a plot graph 1300 showing an example of a mean and boundaries for a SARA reference fitting cluster in a fluid database, as described above with respect to FIGS. 10A-12B. Although NNE predicted field sample concentration of aromatics is close to upper boundary (e.g., cluster sample mean plus 1.5 fold change standard deviation) of the reference data, the predicted concentration of saturates, resins and asphaltanes are generally consistent with the mean values of the compositions over the fluid samples in the fitting cluster. For problematic cases, real-time adaptive model selection or post-processing the data with other relevant models may be used to refine the NNE prediction.

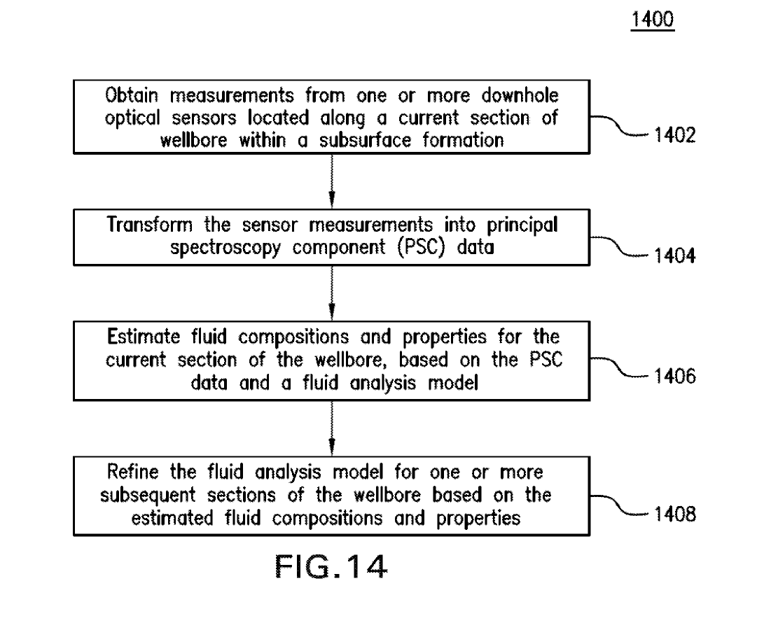

FIG. 14 is a flowchart of an illustrative process 1400 for downhole fluid analysis based on PSC data. For discussion purposes, process 1400 will be described with reference to the illustrative well systems of FIGS. 1A and 1B, as described above. However, process 1400 is not intended to be limited thereto. For example, process 1400 may be implemented in downhole tool 100 for estimating fluid compositions and properties based on measurements obtained from one or more sensors (e.g., optical sensors 52 of FIG. 1A or 1B, as described above) during a downhole operation performed along a wellbore within a subsurface formation.

As shown in FIG. 14, process 1400 begins in block 1402, which includes obtaining measurements from one or more downhole sensors along a current section of wellbore within a subsurface formation. In block 1404, the sensor measurements are transformed into PSC data, as described above. Process 1400 then proceeds to block 1406, which includes estimating at least one fluid composition or property for the current section of the wellbore, based on the PSC data and a fluid analysis model, as described above. In block 1408, the fluid analysis model may be refined for one or more subsequent sections of the wellbore based on the identified fluid types and estimated fluid compositions and properties.

FIG. 15 is a block diagram of an exemplary computer system 1500 in which embodiments of the present disclosure may be implemented. For example, process 1400 of FIG. 14, as described above, may be implemented using system 1500. System 1500 can be a computer, phone, PDA, or any other type of electronic device. In one or more embodiments, such an electronic device may be specially adapted to function as a downhole tool, e.g., downhole tool 100 of FIGS. 1A and 1B, as described above, or component thereof. Such an electronic device includes various types of computer readable media and interfaces for various other types of computer readable media. As shown in FIG. 15, system 1500 includes a permanent storage device 1502, a system memory 1504, an output device interface 1506, a system communications bus 1508, a read-only memory (ROM) 1510, processing unit(s) 1512, an input device interface 1514, and a network interface 1516.

Bus 1508 collectively represents all system, peripheral, and chipset buses that communicatively connect the numerous internal devices of system 1500. For instance, bus 1508 communicatively connects processing unit(s) 1512 with ROM 1510, system memory 1504, and permanent storage device 1502.

From these various memory units, processing unit(s) 1512 retrieves instructions to execute and data to process in order to execute the processes of the subject disclosure. The processing unit(s) can be a single processor or a multi-core processor in different implementations.

ROM 1510 stores static data and instructions that are needed by processing unit(s) 1512 and other modules of system 1500. Permanent storage device 1502, on the other hand, is a read-and-write memory device. This device is a non-volatile memory unit that stores instructions and data even when system 1500 is off. Some implementations of the subject disclosure use a mass-storage device (such as a magnetic or optical disk and its corresponding disk drive) as permanent storage device 1502.

Other implementations use a removable storage device (such as a floppy disk, flash drive, and its corresponding disk drive) as permanent storage device 1502. Like permanent storage device 1502, system memory 1504 is a read-and-write memory device. However, unlike storage device 1502, system memory 1504 is a volatile read-and-write memory, such a random access memory. System memory 1504 stores some of the instructions and data that the processor needs at runtime. In some implementations, the processes of the subject disclosure are stored in system memory 1504, permanent storage device 1502, and/or ROM 1510. For example, the various memory units include instructions for computer aided pipe string design based on existing string designs in accordance with some implementations. From these various memory units, processing unit(s) 1512 retrieves instructions to execute and data to process in order to execute the processes of some implementations.

Bus 1508 also connects to input and output device interfaces 1514 and 1506. Input device interface 1514 enables the user to communicate information and select commands to the system 1500. Input devices used with input device interface 1514 include, for example, alphanumeric, QWERTY, or T9 keyboards, microphones, and pointing devices (also called “cursor control devices”). Output device interfaces 1506 enables, for example, the display of images generated by the system 1500. Output devices used with output device interface 1506 include, for example, printers and display devices, such as cathode ray tubes (CRT) or liquid crystal displays (LCD). Some implementations include devices such as a touchscreen that functions as both input and output devices. It should be appreciated that embodiments of the present disclosure may be implemented using a computer including any of various types of input and output devices for enabling interaction with a user. Such interaction may include feedback to or from the user in different forms of sensory feedback including, but not limited to, visual feedback, auditory feedback, or tactile feedback. Further, input from the user can be received in any form including, but not limited to, acoustic, speech, or tactile input. Additionally, interaction with the user may include transmitting and receiving different types of information, e.g., in the form of documents, to and from the user via the above-described interfaces.

Also, as shown in FIG. 15, bus 1508 also couples system 1500 to a public or private network (not shown) or combination of networks through a network interface 1516. Such a network may include, for example, a local area network (“LAN”), such as an Intranet, or a wide area network (“WAN”), such as the Internet. Any or all components of system 1500 can be used in conjunction with the subject disclosure.

These functions described above can be implemented in digital electronic circuitry, in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself. Accordingly, process 1400 of FIG. 14, as described above, may be implemented using system 1500 or any computer system having processing circuitry or a computer program product including instructions stored therein, which, when executed by at least one processor, causes the processor to perform functions relating to these methods.

As used in this specification and any claims of this application, the terms “computer”, “server”, “processor”, and “memory” all refer to electronic or other technological devices. These terms exclude people or groups of people. As used herein, the terms “computer readable medium” and “computer readable media” refer generally to tangible, physical, and non-transitory electronic storage mediums that store information in a form that is readable by a computer.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., a web page) to a client device (e.g., for purposes of displaying data to and receiving user input from a user interacting with the client device). Data generated at the client device (e.g., a result of the user interaction) can be received from the client device at the server.

It is understood that any specific order or hierarchy of steps in the processes disclosed is an illustration of exemplary approaches. Based upon design preferences, it is understood that the specific order or hierarchy of steps in the processes may be rearranged, or that all illustrated steps be performed. Some of the steps may be performed simultaneously. For example, in certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Furthermore, the exemplary methodologies described herein may be implemented by a system including processing circuitry or a computer program product including instructions which, when executed by at least one processor, causes the processor to perform any of the methodology described herein.

As described above, embodiments of the present disclosure are particularly useful for downhole fluid analysis based on PSC data that is independent of sensor and data type. Accordingly, advantages of the present disclosure include providing a capability for migrating real-time and historical data obtained from various downhole optical tools in diverse oil fields and then, integrating this data with laboratory data into a single generic database for extended applications. By minimizing the number optical inputs that are needed and also, incorporating non-optical inputs, such as fluid density, bubble point and compressibility, the disclosed techniques allow fluid analysis to be performed in real-time for improved, more efficient downhole operations.

Thus, a method for downhole fluid analysis has been described. Embodiments of the method may include: obtaining measurements from one or more downhole sensors along a current section of wellbore within a subsurface formation; transforming the measurements obtained from the one or more downhole sensors into principal spectroscopy component (PSC) data; estimating at least one fluid composition or property for the current section of the wellbore, based on the PSC data and a fluid analysis model; and refining the fluid analysis model for one or more subsequent sections of the wellbore within the subsurface formation, based at least partly on the fluid composition or property estimated for the current section of the wellbore. Further, a computer-readable storage medium has been described. In one or more embodiments, the computer-readable storage medium may have instructions stored therein, which when executed by a computer cause the computer to perform a plurality of functions, including functions to: obtain measurements from one or more downhole sensors along a current section of wellbore within a subsurface formation; transform the measurements obtained from the one or more downhole sensors into principal spectroscopy component (PSC) data; estimate at least one fluid composition or property for the current section of the wellbore, based on the PSC data and a fluid analysis model; and refine the fluid analysis model for one or more subsequent sections of the wellbore within the subsurface formation, based at least partly on the fluid composition or property estimated for the current section of the wellbore.

For any of the foregoing embodiments, the PSC data may be applied as one or more PSC inputs to the fluid analysis model, and the fluid composition or property may be identified based on the applied PSC data. The fluid analysis model may be a neural network having multiple layers of neural network nodes. The one or more downhole sensors may include one or more optical sensors coupled to the downhole tool disposed within the wellbore. Each of the one or more optical sensors may include at least one integrated computational element (ICE) for measuring one or more downhole fluid properties. The one or more downhole sensors may further include one or more non-optical sensors. The one or more non-optical sensors may be selected from the group consisting of: a fluid density sensor: a bubble point sensor; and a compressibility sensor.

Also, for any of the foregoing embodiments, the measurements from the one or more downhole sensors may be transformed based on a reverse transformation model. Accordingly, in one or more embodiments, the method may further include: selecting reference fluids for the reverse transformation model to be calibrated; simulating sensor responses for additional fluids based on a forward transformation model and the selected reference fluids; combining the simulated sensor responses with measured sensor responses of the one or more downhole sensors; and calibrating the reverse transformation model based on the combined simulated and measured sensor responses. Likewise, in one or more embodiments, the plurality of functions performed by the computer when executing instructions stored in the computer-readable storage medium may further include functions to: select reference fluids for the reverse transformation model to be calibrated; simulate sensor responses for additional fluids based on a forward transformation model and the selected reference fluids; combine the simulated sensor responses with measured sensor responses of the one or more downhole sensors; and calibrate the reverse transformation model based on the combined simulated and measured sensor responses.

Also, a system for downhole fluid analysis has been described. Embodiments of the system may include at least one processor and a memory coupled to the processor that has instructions stored therein, which when executed by the processor, cause the processor to perform functions, including functions to: obtain measurements from one or more downhole sensors along a current section of wellbore within a subsurface formation; transform the measurements obtained from the one or more downhole sensors into principal spectroscopy component (PSC) data; estimate at least one fluid composition or property for the current section of the wellbore, based on the PSC data and a fluid analysis model; and refine the fluid analysis model for one or more subsequent sections of the wellbore within the subsurface formation, based at least partly on the fluid composition or property estimated for the current section of the wellbore.

For the foregoing embodiments of the system, the PSC data may be applied as one or more PSC inputs to the fluid analysis model, and the fluid composition or property may be identified based on the applied PSC data. The fluid analysis model may be a neural network having multiple layers of neural network nodes. The one or more downhole sensors may include one or more optical sensors coupled to the downhole tool disposed within the wellbore. Each of the one or more optical sensors may include at least one integrated computational element (ICE) for measuring one or more downhole fluid properties. The one or more downhole sensors may further include one or more non-optical sensors. The one or more non-optical sensors may be selected from the group consisting of: a fluid density sensor; a bubble point sensor; and a compressibility sensor.

In one or more further embodiments of the system, the measurements from the one or more downhole sensors may be transformed based on a reverse transformation model, and the functions performed by the processor may further include functions to: select reference fluids for the reverse transformation model to be calibrated; simulate sensor responses for additional fluids based on a forward transformation model and the selected to reference fluids; combine the simulated sensor responses with measured sensor responses of the one or more downhole sensors; and calibrate the reverse transformation model based on the combined simulated and measured sensor responses.

While specific details about the above embodiments have been described, the above hardware and software descriptions are intended merely as example embodiments and are not intended to limit the structure or implementation of the disclosed embodiments. For instance, although many other internal components of system 1500 are not shown, those of ordinary skill in the art will appreciate that such components and their interconnection are well known.

In addition, certain aspects of the disclosed embodiments, as outlined above, may be embodied in software that is executed using one or more processing units/components. Program aspects of the technology may be thought of as “products” or “articles of manufacture” typically in the form of executable code and/or associated data that is carried on or embodied in a type of machine readable medium. Tangible non-transitory “storage” type media include any or all of the memory or other storage for the computers, processors or the like, or associated modules thereof, such as various semiconductor memories, tape drives, disk drives, optical or magnetic disks, and the like, which may provide storage at any time for the software programming.

Additionally, the flowchart and block diagrams in the figures illustrate the architecture, functionality, and operation of possible implementations of systems, methods and computer program products according to various embodiments of the present disclosure. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The above specific example embodiments are not intended to limit the scope of the claims. The example embodiments may be modified by including, excluding, or combining one or more features or functions described in the disclosure.

As used herein, the singular forms “a”, “an” and “the” are intended to include the plural forms as well, unless the context clearly indicates otherwise. It will be further understood that the terms “comprise” and/or “comprising,” when used in this specification and/or the claims, specify the presence of stated features, integers, steps, operations, elements, and/or components, but do not preclude the presence or addition of one or more other features, integers, steps, operations, elements, components, and/or groups thereof. The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the embodiments in the form disclosed. Many modifications and variations will be apparent to those of ordinary skill in the art without departing from the scope and spirit of the disclosure. The illustrative embodiments described herein are provided to explain the principles of the disclosure and the practical application thereof, and to enable others of ordinary skill in the art to understand that the disclosed embodiments may be modified as desired for a particular implementation or use. The scope of the claims is intended to broadly cover the disclosed embodiments and any such modification.

What is claimed is:

1. A method of downhole fluid analysis, the method comprising:
obtaining measurements from one or more downhole sensors along a current section of wellbore within a subsurface formation;
transforming the measurements obtained from the one or more downhole sensors into principal spectroscopy component (PSC) data;
estimating at least one fluid composition or property for the current section of the wellbore, based on the PSC data and a fluid analysis model; and
refining the fluid analysis model for one or more subsequent sections of the wellbore within the subsurface formation, based at least partly on the fluid composition or property estimated for the current section of the wellbore.

2. The method of claim 1, wherein the PSC data is applied as one or more PSC inputs to the fluid analysis model, and the fluid composition or property is identified based on the applied PSC data.

3. The method of claim 1, wherein the fluid analysis model is a neural network having multiple layers of neural network nodes.

4. The method of claim 1, wherein the one or more downhole sensors include one or more optical sensors coupled to the downhole tool disposed within the wellbore.

5. The method of claim 4, wherein each of the one or more optical sensors includes at least one integrated computational element (ICE) for measuring one or more downhole fluid properties.

6. The method of claim 4, wherein the one or more downhole sensors further include one or more non-optical sensors.

7. The method of claim 6, wherein the one or more non-optical sensors are selected from the group consisting of: a fluid density sensor, a bubble point sensor; and a compressibility sensor.

8. The method of claim 1, wherein the measurements from the one or more downhole sensors are transformed based on a reverse transformation model.

9. The method of claim 8, further comprising:
selecting reference fluids for the reverse transformation model to be calibrated;
simulating sensor responses for additional fluids based on a forward transformation model and the selected reference fluids;
combining the simulated sensor responses with measured sensor responses of the one or more downhole sensors; and
calibrating the reverse transformation model based on the combined simulated and measured sensor responses.

10. A system for downhole fluid analysis, the system comprising:
at least one processor; and
a memory coupled to the processor having instructions stored therein, which when executed by the processor, cause the processor to perform functions including functions to:
obtain measurements from one or more downhole sensors along a current section of wellbore within a subsurface formation;
transform the measurements obtained from the one or more downhole sensors into principal spectroscopy component (PSC) data;
estimate at least one fluid composition or property for the current section of the wellbore, based on the PSC data and a fluid analysis model; and
refine the fluid analysis model for one or more subsequent sections of the wellbore within the subsurface formation, based at least partly on the fluid composition or property estimated for the current section of the wellbore.

11. The system of claim 10, wherein the PSC data is applied as one or more PSC inputs to the fluid analysis model, and the fluid composition or property is identified based on the applied PSC data.

12. The system of claim 10, wherein the fluid analysis model is a neural network having multiple layers of neural network nodes.

13. The system of claim 10, wherein the one or more downhole sensors include one or more optical sensors coupled to the downhole tool disposed within the wellbore.

14. The system of claim 13, wherein each of the one or more optical sensors includes at least one integrated computational element (ICE) for measuring one or more downhole fluid properties.

15. The system of claim 13, wherein the one or more downhole sensors further include one or more non-optical sensors.

16. The system of claim 15, wherein the one or more non-optical sensors are selected from the group consisting of: a fluid density sensor; a bubble point sensor; and a compressibility sensor.

17. The system of claim 10, wherein the measurements from the one or more downhole sensors are transformed based on a reverse transformation model.

18. The system of claim 17, wherein the functions performed by the processor further include functions to:
select reference fluids for the reverse transformation model to be calibrated;
simulate sensor responses for additional fluids based on a forward transformation model and the selected reference fluids;
combine the simulated sensor responses with measured sensor responses of the one or more downhole sensors; and
calibrate the reverse transformation model based on the combined simulated and measured sensor responses.

19. A computer-readable storage medium having instructions stored therein, which when executed by a computer cause the computer to perform a plurality of functions, including functions to:
obtain measurements from one or more downhole sensors along a current section of wellbore within a subsurface formation;
transform the measurements obtained from the one or more downhole sensors into principal spectroscopy component (PSC) data;
estimate at least one fluid composition or property for the current section of the wellbore, based on the PSC data and a fluid analysis model; and
refine the fluid analysis model for one or more subsequent sections of the wellbore within the subsurface formation, based at least partly on the fluid composition or property estimated for the current section of the wellbore.

20. The computer-readable storage medium of claim 19, wherein the fluid analysis model is a neural network having multiple layers of neural network nodes, the PSC data is applied as one or more PSC inputs to the neural network, and the fluid composition or property is identified based on the applied PSC data.
